(19) United States
(12) Patent Application Publication
(10) Pub. No.: US 2008/0288069 A1
(43) Pub. Date: Nov. 20, 2008

(54) Threaded pulley anchor apparatus and method for use in surgical repair of ligament or tendon

(76) Inventors: Alan W. Wolf, Ketchikan, AK (US); Christopher S. Jordan, Midwest City, OK (US)

Correspondence Address: ARTHROCARE CORPORATION, 7500 Rialto Boulevard, Building Two, Suite 100, Austin, TX, 78735-8532, US

(21) Appl. No.: 11/599,138

(22) Filed: Nov. 14, 2006

(51) Int. Cl. A61F 2/08; A61B 17/04

(52) U.S. Cl. 623/13.14; 606/301

(57) ABSTRACT

A cancellous externally threaded pulley anchor for transporting an anterior cruciate ligament graft into the distal femur from the intra-articular surface during a surgical repair of the anterior cruciate ligament. A method using this device eliminates the need to pierce the lateral femoral skin during the surgery and also prevent interference with other surgical instruments and the knee and thigh holder, the anchor providing a pulley for the force insertion of the ligament graft into a femoral bone tunnel after the anchor is installed within the femoral bone tunnel by pulling the unattached end of the suture while the other end of the suture is secured to the ligament graft.

### CROSS REFERENCE TO RELATED APPLICATIONS

None

### I. BACKGROUND OF THE INVENTION

1. Field of Invention

A cancellous externally threaded pulley anchor for transporting an anterior cruciate ligament graft into the distal femur from the intra-articular surface during a surgical repair of the anterior cruciate ligament. A method using this device eliminates the need to pierce the lateral femoral skin during the surgery and also prevent interference with other surgical instruments and the knee and thigh holder, the anchor providing a pulley for the force insertion of the ligament graft into a femoral bone tunnel after the anchor is installed within the femoral bone tunnel by pulling the unattached end of the suture while the other end of the suture is secured to the ligament graft.

2. Description of Prior Art

The following United States patents were discovered and are disclosed within this application for utility patent. All relate to internally affixed surgical anchors. In U.S. Pat. No. 7,083,647 to Sklar, a screw and shank assembly are used to anchor a graft ligament in a bone tunnel, with the shank deployed in the bone tunnel and the screw affixed to the graft installed within the shank. A sleeve and insert are also disclosed in U.S. Pat. No. 5,871,504 to Eaton, wherein the sleeve is inserted into a drilled bone tunnel in the femur and an insert adapted to be threaded within the sleeve is attached to the ligament or graft, which is then installed within the sleeve. U.S. Pat. No. 7,063,717 to St. Pierre discloses an interference fixation device which allows for the passage of a suture attached to a graft to be drawn through the device, the device preventing withdrawal of the suture once installed, retaining the ligament in place.

In U.S. Pat. No. 6,994,725 to Goble, a similar bone tunnel is drilled within the tibial and femur of the knee, but a more narrow passage is drilled in the anterior portion of the femur. Using a C-shaped frame. An externally threaded screw anchor device is placed in the narrow portion with a suture passed through the installed device with the knee appearing to be in a flat orientation during installation. The suture is pulled downward from the bone canal to be attached to a bone graft prepared with what appears to be two plugs, which is then drawn upwards into the bone tunnel.

Other graft anchors are disclosed in U.S. Pat. No. 6,599,289 to Bojarski and U.S. Pat. No. 6,554,553 to Freedland, but do not bear similar elements or installation methods as the current anchor device disclosed herein.

### II. SUMMARY OF THE INVENTION

One of the most common sports injury to the knee involves a tearing of the anterior cruciate ligament in the knee. Over the years, surgery has evolved from open reduction surgical procedures on the knee to reattach the ligament, or what is left of it, between the femur and the tibia. More recently, the surgical procedures have become less intrusive and causing less trauma to the surrounding tissue, resulting in more complete and faster recovery. In the prior art, the most common method involves retaining the knee in a right angle bent position, drilling a hole from the anterior proximal end of the tibia, through the distal anterior portion of the femur, and attaching the ligament or tendon graft to a surgical anchor and installing the surgical anchor within the bone tunnel or just outside the exit hole of the bone tunnel on the anterior surface of the femur. The anchor insertion is terminated when the graft is stretched to its fullest extent, hopefully with a portion inserted within the bone tunnel of the femur.

The present externally threaded anchor device is a cancellous pulley device which is intended to gain purchase or attachment with rotational insertion and allow a pre-placed suture to pull the ACL graft up into a bone tunnel in the distal femur. The intra-articular, femoral sided bone tunnel is drilled through and anterior portion of the proximal tibia or through a puncture hole in the joint capsule, and fully inserted into the bone tunnel of the femur using a tool adapted to a socket in a bottom portion of the device. One end of the suture is sewn or tied into the end of the ACL graft. The opposite end of the suture is then pulled, allowing the device to act as a pulley around which the suture makes a 180 degree turn, wherein the graft end of the suture pulls the ACL graft up into the bone tunnel in the femur into an intended place.

Use of this device prevents the need to puncture the skin of the lateral femur with an extended delivery device, reducing the chance of contamination or infection during and after surgery as well as eliminating an extra puncture wound. It also eliminates the potential of interference with the tourniquet and thigh-holder used during the surgical procedure during installation of the device.

The primary objective of the invention is to provide an anchor utilized in a surgical ACL repair procedure which is pre-threaded with a suture loop attached within a bone tunnel through the tibia into the femur which will act as a pulley for the suture, attaching one end of the suture to a ligament graft and pulling on the other end of the suture to draw the ligament graft into the bone tunnel and subsequently tying off the free end of the suture when the ligament graft is at the appropriate location and tension chosen by the surgeon. A second objective is to reduce the potential of infection, contamination or interference with other surgical instruments and appliances during the ACL surgical procedure by eliminating unnecessary puncture wounds in the skin, especially the outer skin over the distal end of the femur.

### III. DESCRIPTION OF THE DRAWINGS

The following drawings are submitted with this utility patent application.

FIG. 1 is a side view of the first embodiment of the threaded pulley anchor device.

FIG. 2 is an end view of the device along section lines 2/2 of FIG. 1.

FIG. 3 is a side cross-sectional view of the device along section lines 3/3 of FIG. 1.

FIG. 4 is view of the device being inserted through a bone tunnel in the upper anterior portion of the tibia into the lower posterior portion of the femur using a tool with the sutures attached to the device.

FIG. 5 is a view of the device installed within the bone tunnel of the lower anterior portion of femur with one end of the suture attached to the ligament or tendon graft with the free end of the suture extending through the bone tunnel to draw the ligament or tendon graft into the bone tunnel in the femur.

FIG. 6 is a side view of the second embodiment of the threaded pulley anchor device.

FIG. 7 is an end view of the device along section lines 7/7 of FIG. 6.

FIG. 8 is a lower view of the device along section lines 8/8 of FIG. 6.

FIG. 9 is a side cross sectional view of the device along section lines 9/9 of FIG. 8.

### IV. DESCRIPTION OF THE PREFERRED EMBODIMENT

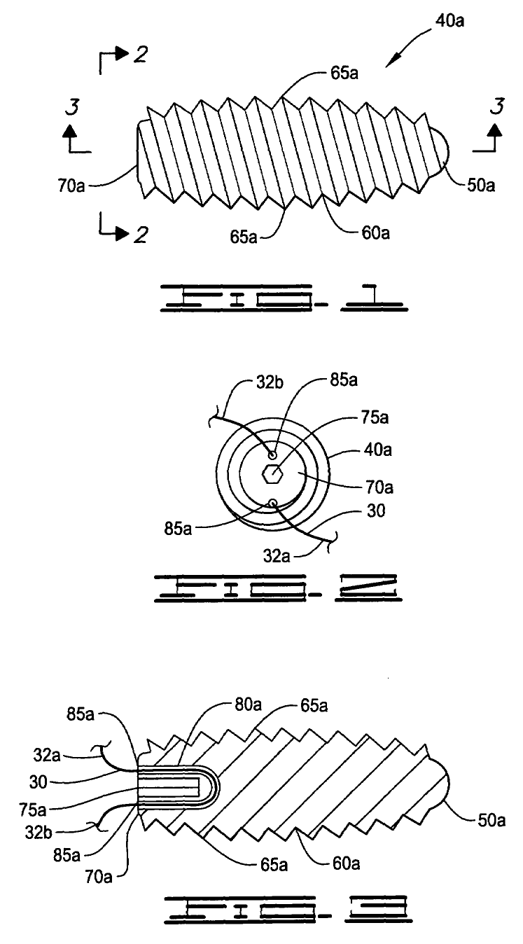

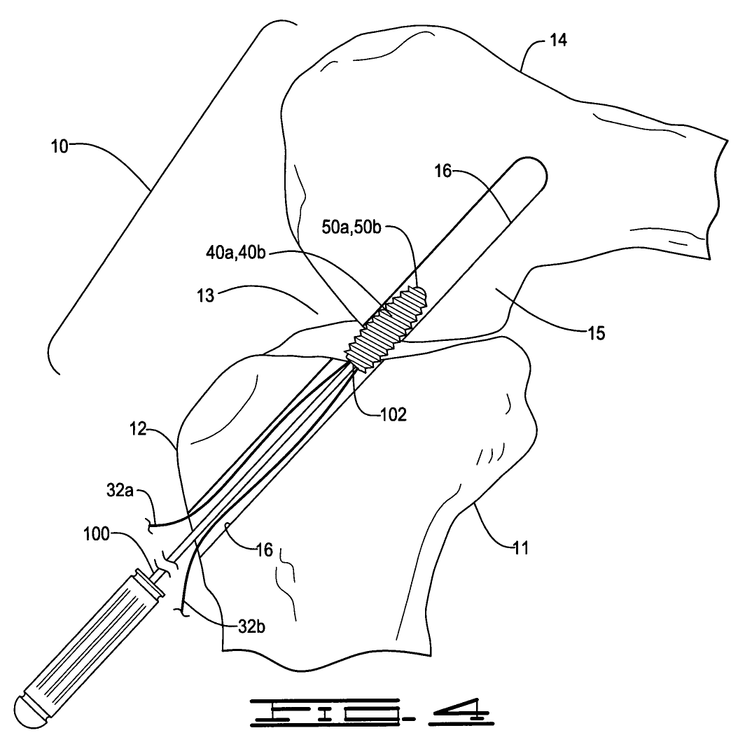

An externally threaded pulley anchor device 40a, 40b, shown in FIGS. 1-3 of the drawings, is utilized in the surgical repair of a ligament in a joint, specifically an anteriorcruciate ligament in the knee, a first embodiment of the device 40a comprising a tapered first end 50a, a body member 60a defining an external thread 65a and a flattened second end 70a, FIGS. 1 and 3, a recessed central socket slot 75a in the second end 70a, FIG. 2, and an arch-shaped suture passage 80a within the body member 60a adapted to receive a surgical suture 30, the suture passage 80a extending into the body member 60a around the central socket slot 75a, FIG. 3, the suture passage 80a having two external suture openings 85a in the second end 70a on opposite sides of the central socket slot 75a, FIG. 2, wherein the suture 30 is passed through the suture passage 80a prior to surgical application with two ends 32a, 32b extending from the external suture openings 85a.

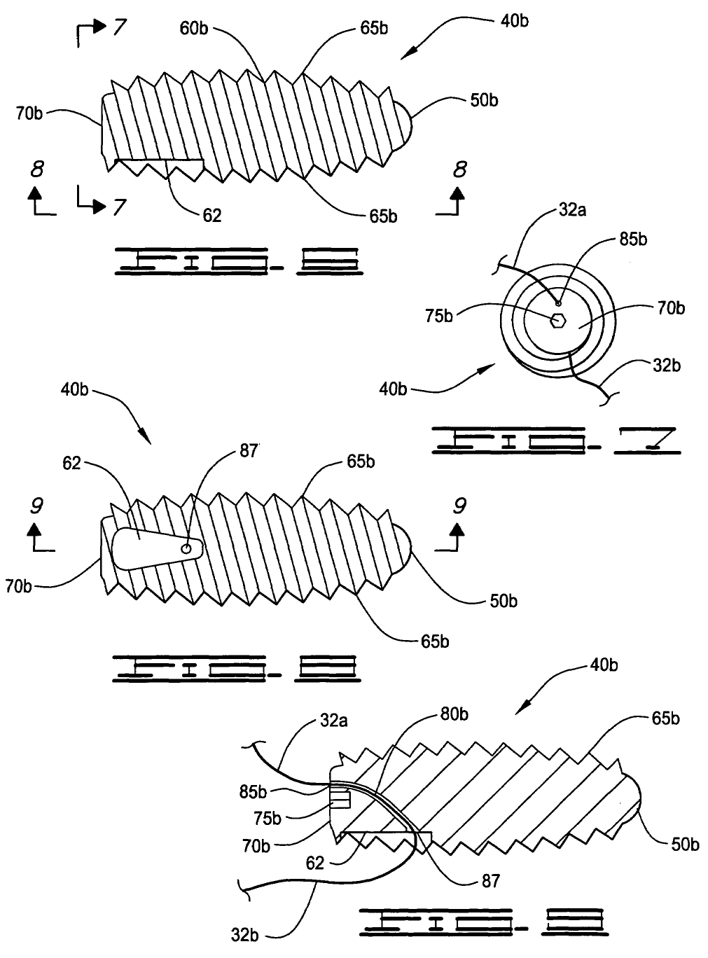

A second embodiment of the device 40b, shown in FIGS. 6-9 of the drawings, comprises a tapered first end 50b, a body member 60b defining an external thread 65b, a flat side segment 62 and a flattened second end 70b having a recessed central socket slot 75b, FIGS. 7 and 9, and a suture passage 80b within the body member 60b adapted to receive a surgical suture 30, the suture passage 80b entering the flattened second end 70b at a first external suture opening, continuing around the central socket slot 75a, and terminating at a second external suture opening 87 in the flat side segment 62, FIGS. 8 and 9, wherein the suture 30 is passed through the suture passage 80b prior to surgical application with two ends 32a, 32b extending from the respective first and second external suture openings, FIGS. 7 and 9. Most preferably, the first and second embodiments of the device are a cancellous biocompatible material which is suitable for being permanently placed within a bone in the human body.

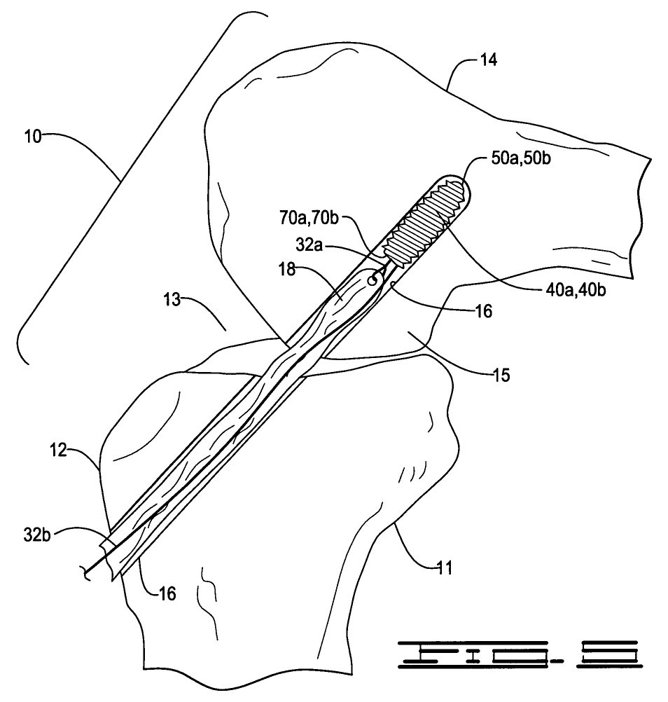

Although having the potential for reattachment of any ligament in a joint, the device as utilized in an ACL surgical repair would require surgical preparation of the knee 10, including the placement of the knee 10 at a ninety degree angle with the leg retained in a thigh-holder. A tourniquet is applied above the knee. After the skin is properly cleaned and sterilized, an incision is made in the skin covering the upper anterior portion 12 of the tibia 11, and a bone tunnel 16 is drilled from the upper anterior portion 12 of the tibia 11 into the distal end 15 of the femur 14 through the knee capsule 13, FIGS. 4 and 5. After debridement of the bone tunnel 16, with the suture pre-threaded within the device 40a, 40b, the tapered first end 50a, 50b of the device is inserted into the bone tunnel 16 in tibia 11 and screwed into the bone tunnel 16 using an elongated tool 100 having an end 102 adapted to the central socket slot 75a, 75b to rotate and insert the device into the bone tunnel 16 in the femur 14 as the external thread 65a, 65b gains purchase within the bone tunnel 16 as the device 40a, 40b is advanced into the femur 14, FIG. 4. The bone tunnel 16 in the femur 14 does not extend through the entire distal end 15 of the femur 14 in this application, FIGS. 4 and 5. Once the device 40a, 40b is fully inserted within the bone tunnel 16 of the femur 14, one end 32a of the pre-threaded suture 30 is attached and affixed to a free end of an ACL graft 18. After attachment to the ACL graft 18, the unattached end 32b of the suture 30 is then pulled from the bone tunnel 16 drawing the ACL graft 18 into the bone tunnel 16 in the femur 14, the device 40a, 40b acting as a pulley upon the suture 30, FIG. 5. After the ACL graft 18 is drawn to an optimal tension and length within the bone tunnel 16 in the femur 14, the unattached end 32b of the suture 30 is tied off, either to the ACL graft 18 or to another suitable structure to maintain tension on the ACL graft 18. Any excess length of suture 30 is trimmed off and the external wound or incision is closed.

While the invention has been particularly shown and described with reference to a preferred embodiment thereof, it will be understood by those skilled in the art that changes in form and detail may be made therein without departing from the spirit and scope of the invention.

What is claimed is:

1. An externally threaded pulley anchor device utilized in the surgical repair of a ligament in a joint, specifically an anterior cruciate ligament in a knee, the device comprising:
a tapered first end, a body member defining an external thread and a flattened second end;
a recessed central socket slot in said second end; and
an arch-shaped suture passage within said body member adapted to receive a surgical suture having two ends, said suture passage extending into said body member around said central socket slot, said suture passage having two external suture openings in said second end with said central socket slot between said suture openings, wherein a suture is installed within said suture passage with said suture ends extending out respective said suture openings prior to installation of the device.

2. The device as disclosed in claim 1 wherein said device is a biocompatible, cancellous material suitable for permanent placement within a bone in the human body

3. A method for the surgical repair of the anterior cruciate ligament in a knee joint comprising the steps of:
a. positioning said knee joint at a ninety degree angle;
b. preparing said knee for surgery by sterilization of said knee;
c. drilling a bone tunnel from an upper anterior portion of a tibia through a knee capsule and partially within a distal end of a femur without penetration into a lateral femoral cortex of said femur;
d. inserting a tapered first end of an externally threaded pulley anchor device into the bone tunnel in said tibia, said pulley anchor device further comprising a body member defining an external thread and a flattened second end, a recessed central socket slot in said second end, and an arch-shaped suture passage within said body member adapted to receive a surgical suture having two ends, said suture passage extending into said body member around said central socket slot, said suture passage having two external suture openings in said second end with said central socket slot between said suture openings, wherein said surgical suture is installed within said suture passage with said suture ends extending from respective said suture openings prior to installation of the device;
e. installing said device within said bone tunnel using a tool having an end adapted to said recessed central socket slot to rotate said device, causing said external thread of said device to gain purchase within said bone tunnel while advancing said device within said bone tunnel of said femur;
f. securing one said end of said suture an ACL graft in said knee attached to said tibia;
g. pulling said unattached end of said suture from said bone tunnel while drawing the ACL graft into said bone tunnel, said device acting as a pulley upon said suture;
h. tying off said unattached end of said suture to said ACL graft or other suitable structure within said knee joint.

4. An externally threaded pulley anchor device utilized in the surgical repair of a ligament in a joint, specifically an anterior cruciate ligament in a knee, the device comprising:
a tapered first end;
a body member defining an external thread, a flat side segment and a flattened second end having a recessed central socket slot; and
a suture passage within said body member adapted to receive a surgical suture, said suture passage entering said flattened second end at a first external suture opening, continuing around said central socket slot, and terminating at a second external suture opening in said flat side segment, wherein said suture is passed through said suture passage prior to surgical application with two ends of said suture extending from said respective first and second external suture openings.

5. The device as disclosed in claim 4 wherein said device is a biocompatible, cancellous material suitable for permanent placement within a bone in the human body

6. A method for the surgical repair of the anterior cruciate ligament in a knee joint comprising the steps of:
a. positioning said knee joint at a ninety degree angle;
b. preparing said knee for surgery by sterilization of said knee;
c. drilling a bone tunnel from an upper anterior portion of a tibia through a knee capsule and partially within a distal end of a femur without penetration into a lateral femoral cortex of said femur;
d. inserting a tapered first end of an externally threaded pulley anchor device into said bone tunnel in said tibia, said pulley anchor device further comprising a tapered first end, a body member defining an external thread, a flat side segment and a flattened second end having a recessed central socket slot, and a suture passage within said body member adapted to receive a surgical suture, said suture passage entering said flattened second end at a first external suture opening, continuing around said central socket slot, and terminating at a second external suture opening in said flat side segment, wherein said suture is passed through said suture passage prior to surgical application with two ends of said suture extending from said respective first and second external suture openings;
e. installing said device within said bone tunnel using a tool having an end adapted to said recessed central socket slot to rotate said device, causing said external thread of said device to gain purchase within said bone tunnel while advancing said device within said bone tunnel of said femur;
f. securing one said end of said suture an ACL graft in said knee attached to said tibia;
g. pulling said unattached end of said suture from said bone tunnel while drawing the ACL graft into said bone tunnel, said device acting as a pulley upon said suture;
h. tying off said unattached end of said suture to said ACL graft or other suitable structure within said knee joint.
